(19) United States
(12) Plant Patent Application Publication
(10) Pub. No.: US 2010/0199397 P1
(43) Pub. Date: Aug. 5, 2010

(54) Poinsettia plant named 'LAZZPO1047'

(50) ;

(76) Inventor: Valentino Lazzeri, Merano (IT)

Correspondence Address: John Alumit, 16830 Ventura Blvd. Suite 360, Encino, CA, 91436, US

(21) Appl. No.: 12/590,316

(22) Filed: Nov. 6, 2009

(30) Foreign Application Priority Data
Feb. 4, 2009 (IT) 20090193

(51) Int. Cl. A01H 5/00

(52) U.S. Cl. PLT/307

(57) ABSTRACT

A new and distinct Poinsettia ‘LAZZPO1047’ particularly described by combining features of optimum branching and uniformity of growth. Medium sized dark green foliage, medium strong growth, moderate to zero need for growth retardants depending on desired pot size and growth conditions. The bract is an intense red color, medium sized inflorescence, and does not hide all the leaves below so that there is a nice contrast between the red bract and some visible leaves. Slightly wavy petal along the mid riff. The border of some petals is slightly dark violet. Good cyathia. Middle, early flowering response for the beginning of the December market. Toward the end of cultivating, it can be grown at 15° C., optimum shelf life, perfect for “mass production”, easy to grow because of good branching abilities.

### CROSS-REFERENCE TO RELATED APPLICATION

This patent application claims the benefit of Community Plant application no. 20090193 filed with the Community Plant Variety Office (CPVO) on Feb. 4, 2009.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### LATIN NAME OF THE GENUS AND SPECIES

Euphorbia pulcherrima Wild. ex Klotzsch.

### VARIETY DENOMINATION

LAZZPO1047

### BACKGROUND

The present invention relates to a new and distinct cultivar of Poinsettia plant botanically known as Euphorbia pulcherrima Willd. ex Klotzsch; hereinafter referred to by the variety denomination ‘LAZZPO1047’.

The new cultivar is a product of a planned breeding program with the objective of creating new Poinsettia cultivars with a red flower color in combination with dark-green foliage and good cultivation ability. ‘LAZZPO1047’ was originated from a hybridization made in Merano, Italy in spring 2005.

The female parent was the Poinsettia cultivar ‘Eckacton’ (market name: Freedom Orange), characterized by an orange-red bract, early flower and dark-green foliage, with good resistance to conditions not as suitable for growing as with other Poinsettias. The male parent was the Poinsettia cultivar ‘Eckakeem’ (market name: Primero Red), characterized by very good branching, a tendency to form horizontal branches, dark-green foliage, intense red bract, with a slight bluish color, and late flower.

The seeds obtained from this hybridization were germinated in the summer of 2005, and the resulting seedlings were selected during their flowering period in December 2005. In the spring of 2006, cuttings were taken from a single plant. These cuttings were grafted onto rootstocks of the variety ‘Beckmann's Altrosa’ (U.S. Plant Pat. No. 9,336), also known as ‘Maren’, in order to induce better branching ability. From the upper area of the successfully grafted plants, shoot tip cuttings were taken for the cultivation of branched plants for the second examination in the fall of 2006. The trial was repeated in 2007 and 2008.

Horticultural examination of the clone starting in 2006 and continuing thereafter has confirmed that the combination of characteristics as herein disclosed for ‘LAZZPO1047’ are firmly fixed and retained through successive generations of asexual reproduction.

The following traits have been repeatedly observed and are determined to be the basic characteristics of ‘LAZZPO1047’ which, in combination, distinguish this Poinsettia as a new and distinct cultivar: optimum branching and uniformity of growth, medium sized dark green foliage, medium strong growth, moderate to zero need for growth retardants, depending on the desired pot size and growth conditions, intense red bract, medium sized inflorescence, which does not hide all the leaves below, so that there is a nice contrast between the red bract and some visible leaves, good cyathia, middle early flowering response for the beginning of the December market, and optimum shelf life.

This new Poinsettia plant is illustrated by the accompanying photographs which show the plant form, foliage and flowers. The colors shown are as true as can be reasonably obtained by conventional photographic procedures.

### SUMMARY

A new and distinct cultivar of Poinsettia plant botanically known as Euphorbia pulcherrima Willd. ex Klotzsch, and referred to by the variety denomination ‘LAZZPO1047’.

The female parent was the Poinsettia cultivar ‘Eckacton’ (market name: Freedom Orange), and the male parent was the Poinsettia cultivar ‘Eckakeem’ (market name: Primero Red).

The following traits have been repeatedly observed and are determined to be the basic characteristics of ‘LAZZPO1047’ which, in combination, distinguish this Poinsettia as a new and distinct cultivar: optimum branching and uniformity of growth, medium sized dark green foliage, medium strong growth, moderate to zero need for growth retardants, depending on the desired pot size and growth conditions, intense red bract, medium sized inflorescence, which does not hide all the leaves below, so that there is a nice contrast between the red bract and some visible leaves, good cyathia, middle early flowering response for the beginning of the December market, and optimum shelf life.

### FIGURES

FIG. 1 shows one pinched plant in pot, 13 cm with its flowers, branches and leaves.

FIG. 2 shows one flower in detail

FIGS. 3 and 4 are plants from the growth test at Merano (13 cm pot, potted in week 31/2008, pinched in week 33/2008, grown in a normal production greenhouse) at full bloom in week 52/2008. They have been sleeved and then boxed. The boxes were kept for 3 days in an dark freezer at 15° C. to simulate transport condition. Then they were put into a room with low light conditions for the next six weeks to simulate endconsumer conditions. FIG. 3 shows six plants in the first repetition at the end of the shelf life test on Feb. 6, 2009. FIG. 4 shows six plants in the second repetition at the end of the shelf life test on Feb. 6, 2009.

FIG. 5 shows twelve plants in a 11 cm pot, sleeved and put into a watertray on Nov. 4, 2008. The plants were grown in Sabaudia, Italy. The cuttings were directly placed in an 11 cm pot on week 32/2008. After five weeks, they were pinched. During cultivation, they were sprayed lightly with growth retardant CCC at 1% to obtain more regular growth.

FIGS. 6 and 7 show the plants with 0×CCC (not treated) and 2×CCC, respectively. To see the effect of the growth retardant CCC in 2007, at our location in Merano, Italy, the plants were pinched in 13 cm pots, treated by leave spray of 1% CCC to see the effect on the growth. There were 4 variants: 0×CCC, 1×CCC, 2×CCC, 4×CCC

FIGS. 8 through 12 show pictures of the plant from weeks 41 through 47 in 2008. FIGS. 13 through 18 show pictures of the plant from weeks 40 through 47 in 2007. FIGS. 19 through 23 show pictures of the plant from weeks 43 through 48 in 2006. In Merano, Italy, in a normal production greenhouse growth and examination tests are conducted every year. Plants were potted in 13 cm pots at week 31 and pinched in week 33. From week 40 on, pictures were taken of the plants to document flower development.

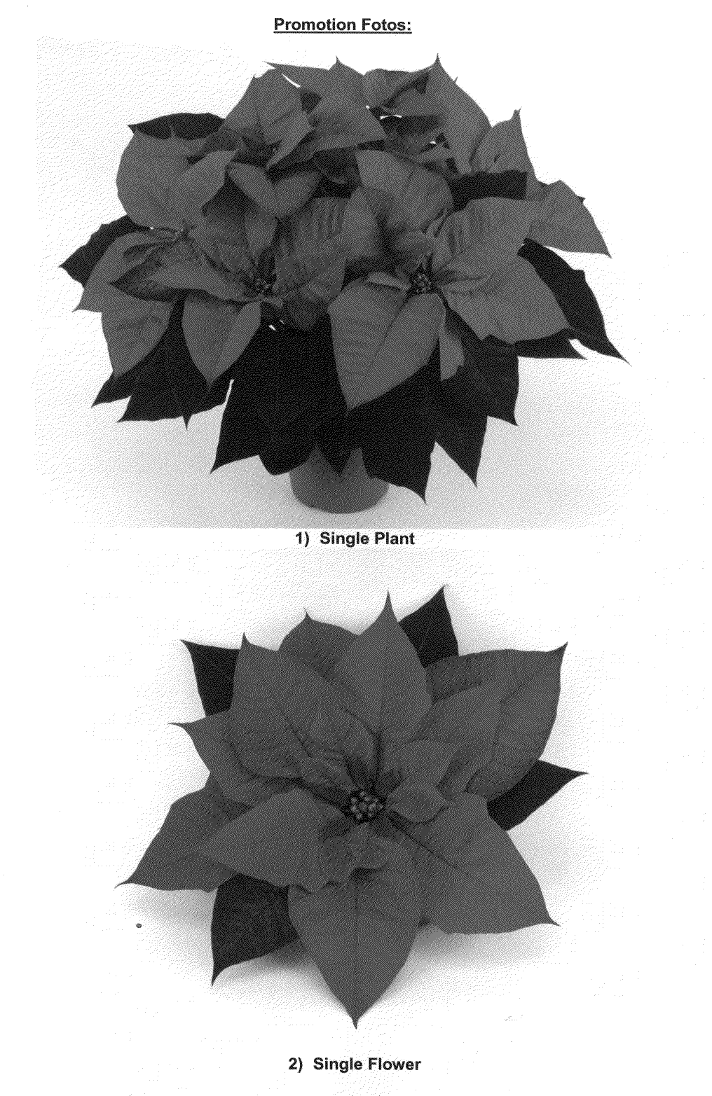

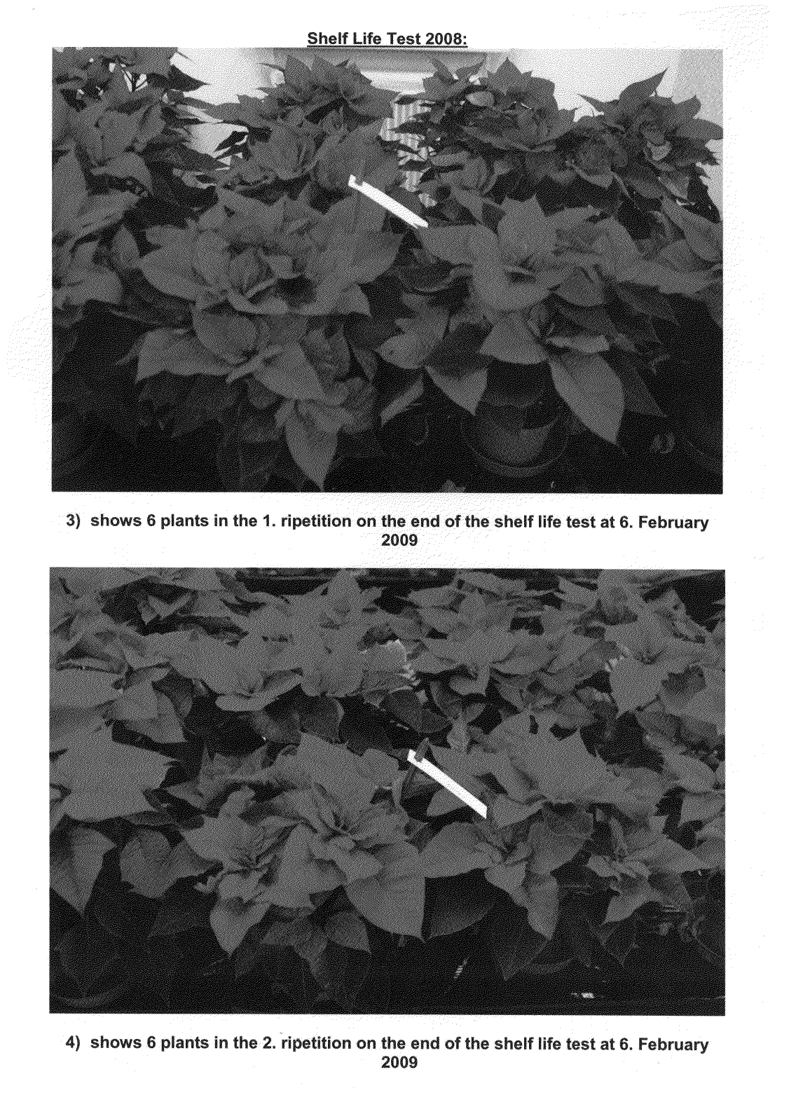

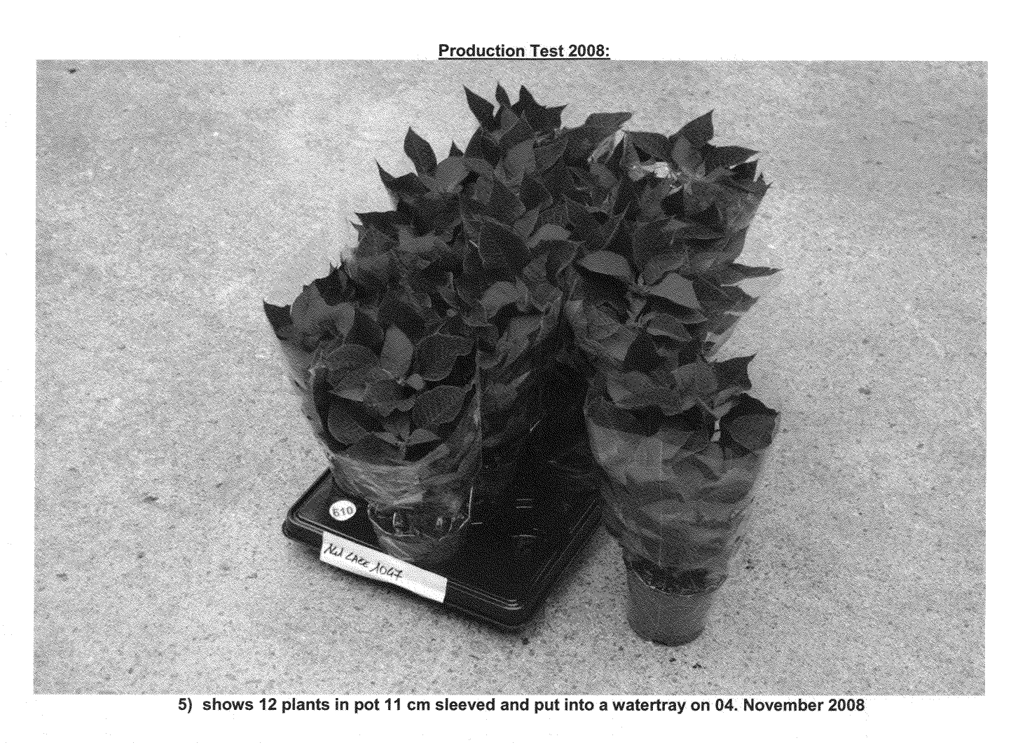

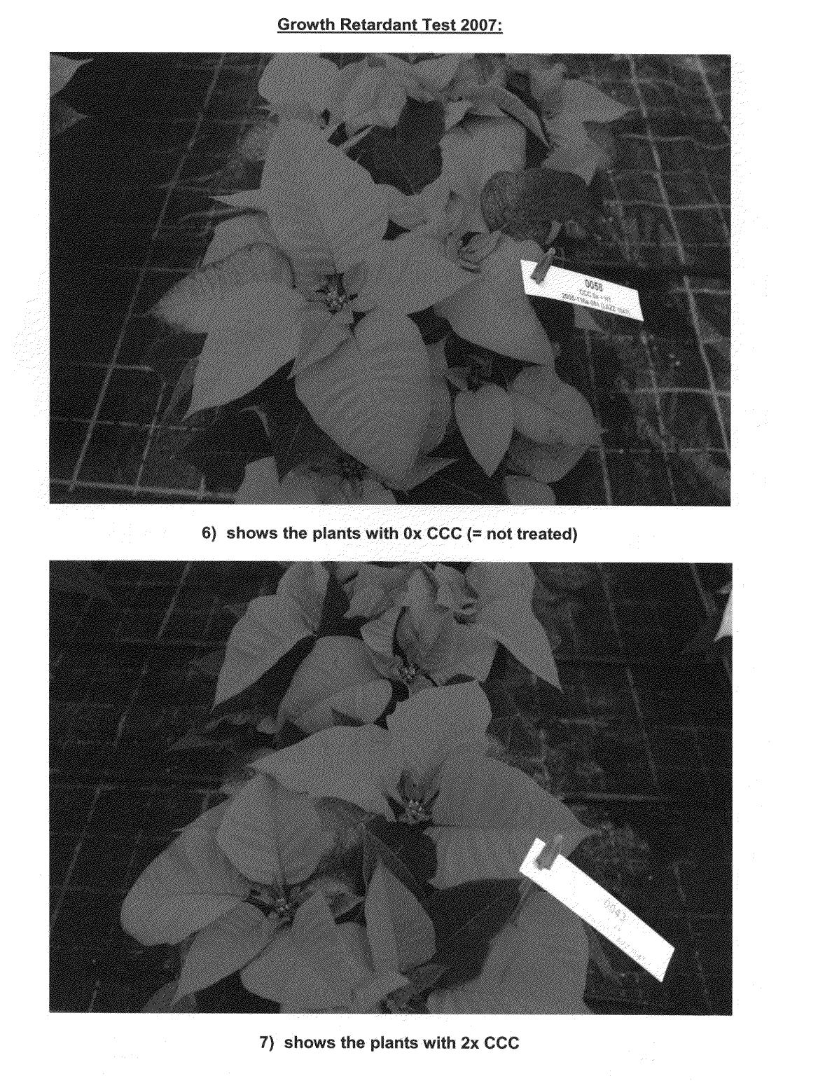

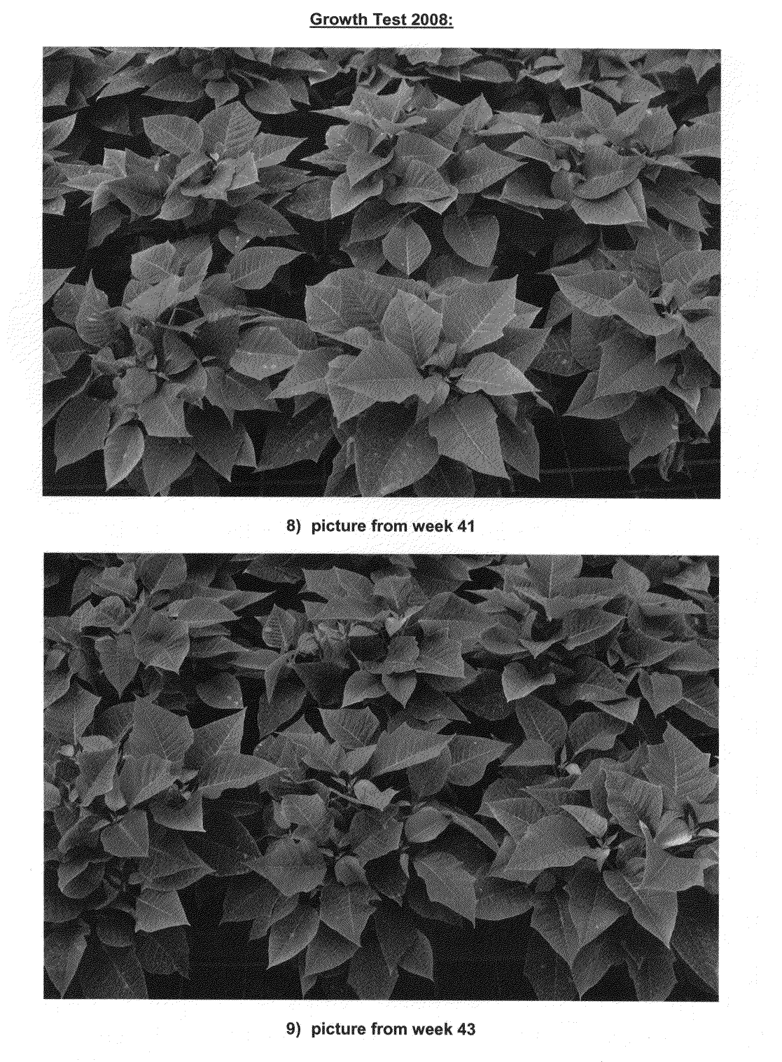

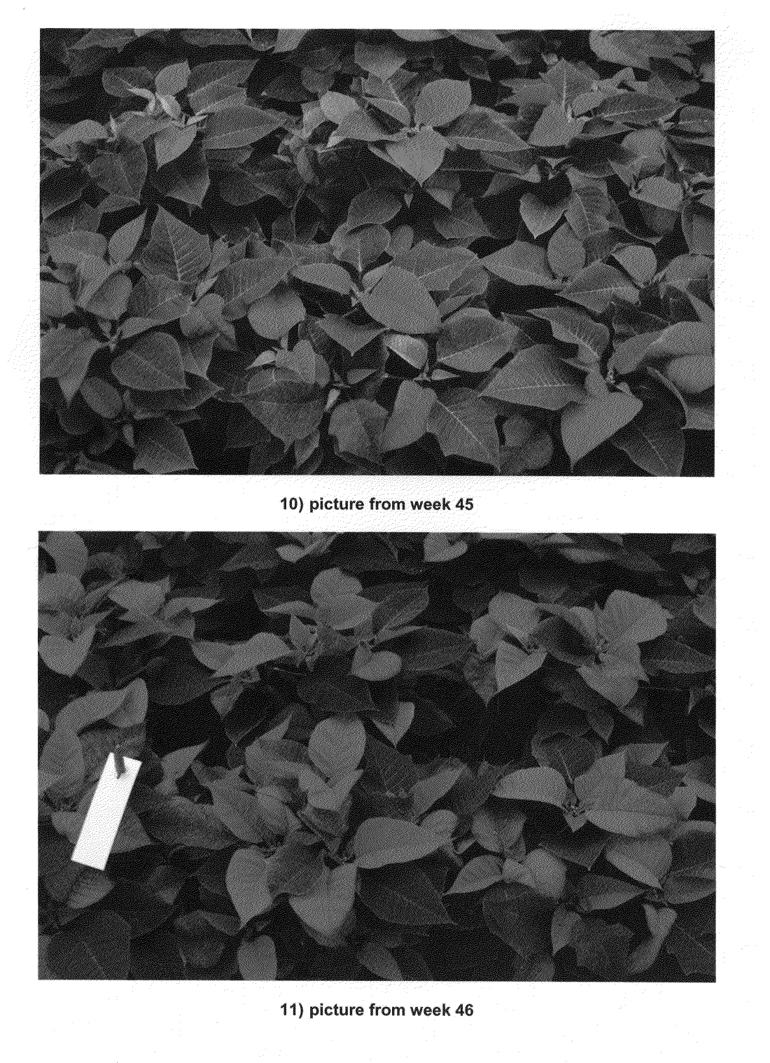

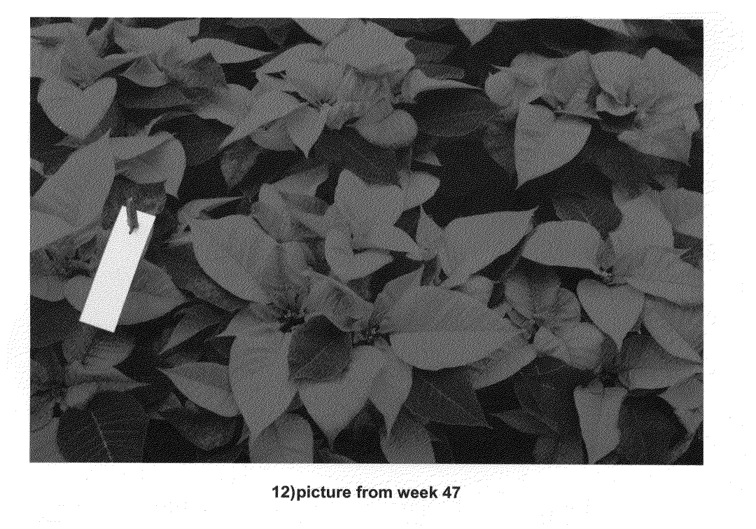

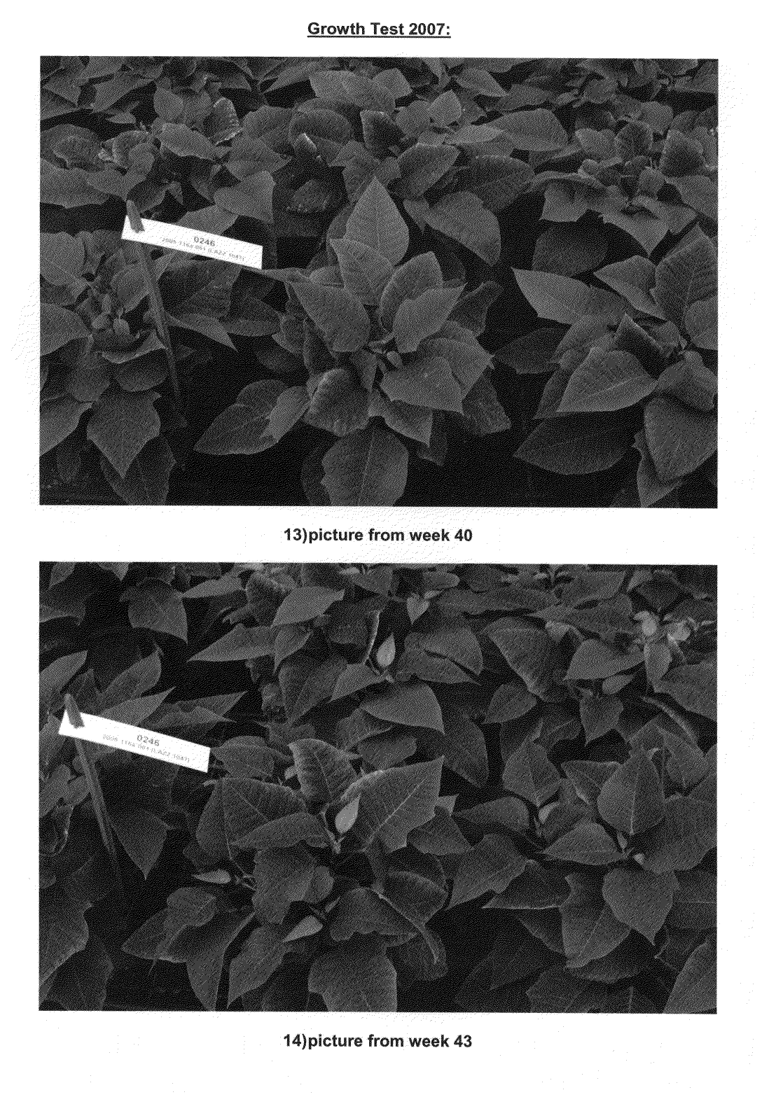

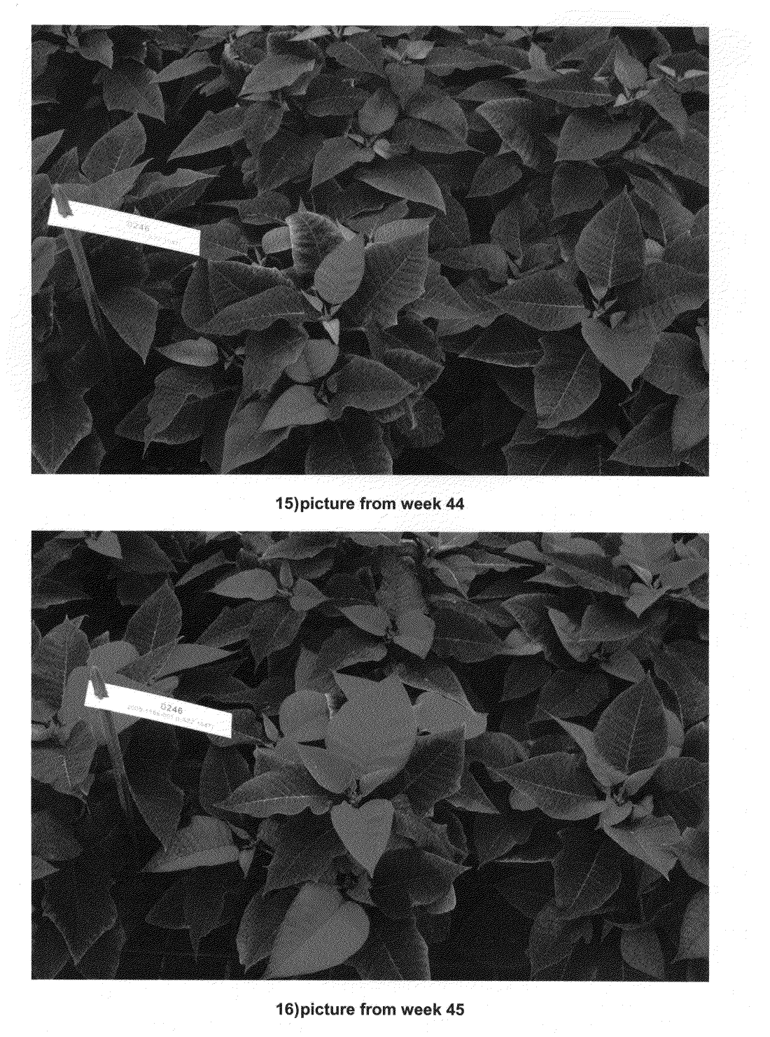

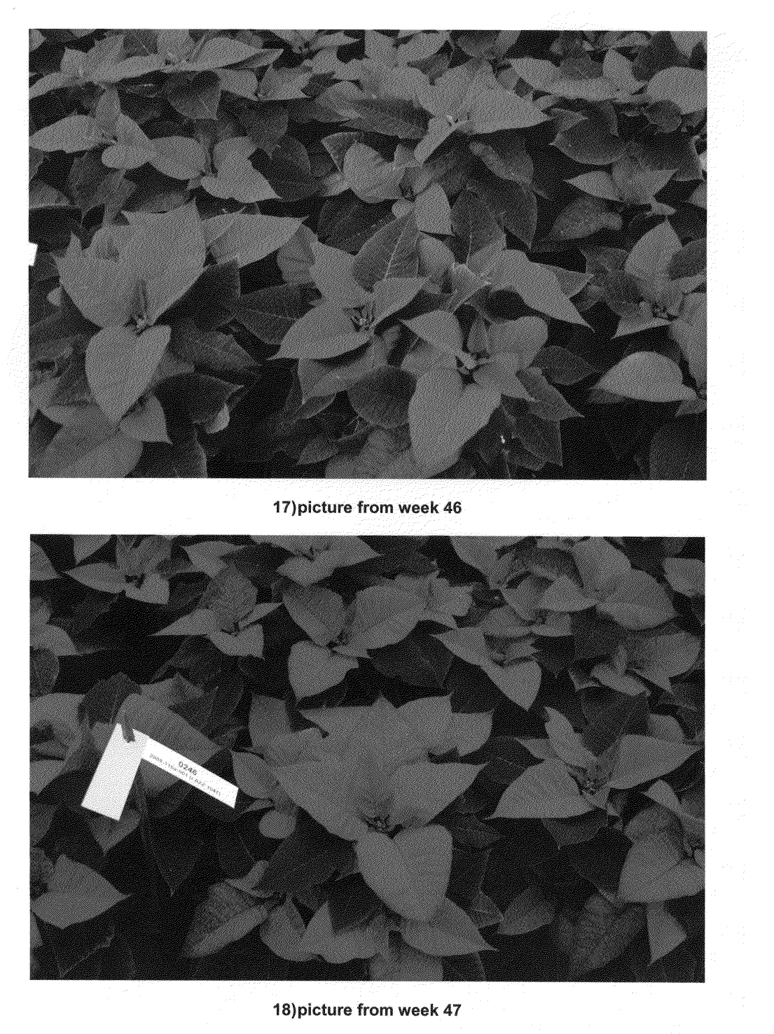

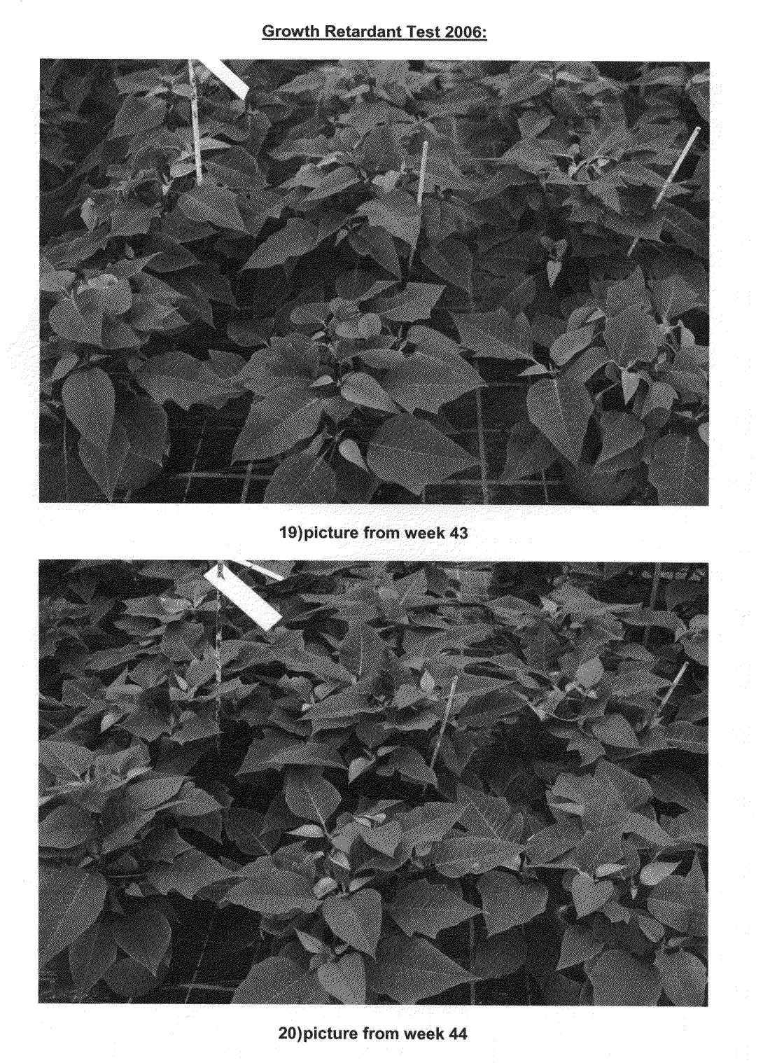

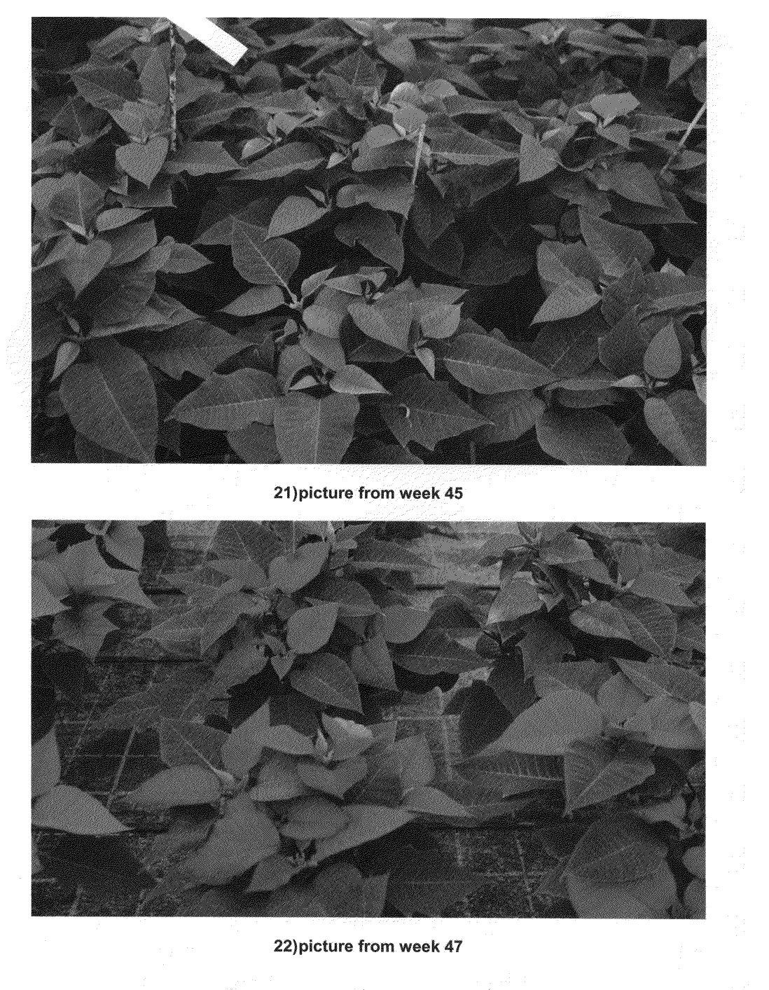

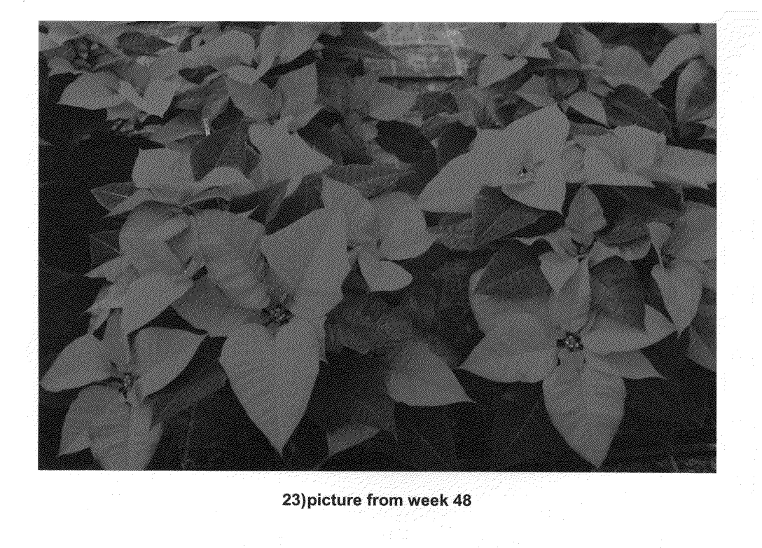

### DESCRIPTION

DESCRIPTION OF THE NEW CULTIVAR. ‘LAZZPO1047’ has not been observed under all possible environmental conditions. The phenotype may vary significantly with variations in environment such as temperature, light intensity, and day-length. The following observations, measurements and comparisons describe the plants grown in Merano, Italy under greenhouse conditions which approximate those generally used in commercial practice.

The plants described herein were grown in a greenhouse in Merano, Italy from the summer to the fall of 2008. Rooted cuttings were planted in 13 cm pots on Jul. 29, 2008, and were pinched on Aug. 11, 2008, which left 7-8 leaves remaining. Initially, the minimum temperature was 18° C. Beginning on Sep. 15, 2008, the temperature was lowered to a minimum temperature of 16° C. The plants initiated flowers under natural short-day conditions in the fall. No black cloth was applied to the greenhouse to simulate short-day conditions. No growth regulator was applied.

Observations and measurements were mainly taken in early to mid-December 2008 when the plants were in full bloom and about 20 weeks old. In the following description, color references are made to The Royal Horticultural Society Colour Chart (R.H.S.).
Description of the new plant: Classification: Family. — Euphorbiaceae. Botanical. — Euphorbia pulcherrima (Willd Ex Klotzsch). Parentage: Female parent. — Poinsettia cultivar ‘Eckacton’ (market name: Freedom Orange). Male parent. — Poinsettia cultivar ‘Eckakeem’ (market name: Primero Red). Growth: Time to produce a rooted cutting. — about 21 days in a greenhouse at a temperature of 22-24° C. Blooming habit. — Beginning under natural short-day conditions in fall: Botanically (cyathia open): begin of week 48, end of November. Commercially (bracts colored, marketable): from week 48, end of November. Flower response time. — About 8 weeks. Flowering season. — Mainly from late November to late December. Keeping quality. — Very good, better or as good like the best keeping varieties on the market at moment. Plant: Form. — shrub, self-branching. Growth habit. — Medium vigor, compact structure, pinched plants are bushy with the branches upright-directed at an angle of roughly 45°; foliage canopy uniformly rounded. Height in 13 cm pot. — 23.0 cm as measured from soil line. Width. — 39.5 cm. Average number of branches. — 7.8. Length of branches. — 17-23 cm. Average number of inflorescences. — 7.5. Leaves: Shape. Ovate to light dented. Inflorescence with surrounding bracts. — Medium sized, horizontally — with the bract tip slightly downward-directed, bract surface is slightly waved. Cyathia number. — 5-9 born in a tight cluster. Nectar cups. — yellow. Seeds. — None observed. Disease and insect resistance: No observation made.
Comparison with known cultivars: The most similar cultivars in comparison to ‘LAZZPO1047’ are the varieties ‘Eckakeem’ (market name: Primero Red) and ‘Duepre’ (market name: Premium Red). In comparison to ‘Eckakeem’, ‘LAZZPO1047’ has less bluish red bract, is earlier in bloom, has less strong growth with less tendency for horizontal branches. In comparison to ‘Duepre’, ‘LAZZPO1047’ is one week later in bloom and has a more bluish red bract.

What is claimed is:

1. A new and distinct Poinsettia plant named ‘LAZZPO1047’ as described and illustrated herein.
